(19) United States
(12) Patent Application Publication
(10) Pub. No.: US 2016/0022772 A1
(43) Pub. Date: Jan. 28, 2016

(54) Cytokine Receptors as Targets for Hypertension Therapy and Methods of Use

(71) Applicant: Duke University, Durham, NC (US)

(72) Inventors: Steven D. Crowley, Durham, NC (US); Jian-dong Zhang, Cary, NC (US)

(21) Appl. No.: 14/811,310

(22) Filed: Jul. 28, 2015

Related U.S. Application Data
(60) Provisional application No. 62/029,702, filed on Jul. 28, 2014.
(60) Provisional application No. 62/061,551, filed on Oct. 8, 2014.
(60) Provisional application No. 62/161,979, filed on May 15, 2015.

(51) Int. Cl. A61K 38/17

(52) U.S. Cl. CPC A61K 38/1793

(57) ABSTRACT

The present disclosure provides methods of treating or ameliorating hypertension in a subject comprising administering a therapeutically effective amount of an IL-1 receptor antagonist.

### CROSS-REFERENCE TO RELATED APPLICATIONS

This application claims the benefit of the filing dates of U.S. provisional application No. 62/029,702, filed Jul. 28, 2014, U.S. provisional applications No. 62/061,551, filed Oct. 8, 2014, and U.S. provisional application No. 62/161,979, filed May 15, 2015, the disclosures of each are incorporated by reference in their entirety.

### STATEMENT REGARDING FEDERALLY SPONSORED RESEARCH

This invention was made with Government support under Federal Grant No. DK087893 awarded by the National Institutes of Health. The Government has certain rights to this invention

### BACKGROUND OF THE INVENTION

1. Field of the Invention

This invention generally relates to methods for treating or ameliorating hypertension.

2. Description of Related Art

Hypertension impacts one billion people worldwide and leads to catastrophic cardiovascular complications including heart failure, stroke, and chronic kidney disease. Nevertheless, following the development more than 20 years ago of agents that inhibit the renin-angiotensin system (RAS), novel blood pressure-lowering therapies have not emerged. This failure to develop new anti-hypertensive agents is a major public health concern given that blood pressure remains poorly controlled in large numbers of hypertensive patients.

### SUMMARY OF THE INVENTION

The macrophage cytokine interleukin 1 (IL-1) is a key regulator of innate immunity. The inventors have discovered that IL-1 has a role in atherosclerosis. The inventors have also advantageously discovered that expression of both IL-1 isoforms, IL-1alpha and IL-1 beta, are elevated in the kidney during hypertension (Crowley et al., Hypertension 2010; 55:99). Therefore, the activation of the IL-1 receptor potentiates blood pressure elevation. For example, the inventors have found that IL-1 receptor activation suppresses the accumulation of nitric-oxide (NO) producing myeloid derived suppressor cells (MDSC) in the kidney, leading to augmented renal salt retention via the NKCC2 sodium co-transporter in the thick ascending limb of the nephron. Accordingly, IL-1 receptor blockade, by acting upstream of NO and NKCC2 in this cascade, had a profound blood pressure-lowering effect.

Therefore, in one aspect, the disclosure provides methods for treating or ameliorating hypertension comprising, consisting of, or consisting essentially of, administering an IL-1 receptor antagonist at a therapeutically effective level to a subject with elevated blood pressure.

In one aspect, the disclosure provides methods for treating or ameliorating hypertension comprising administering Anakinra at a therapeutically effective level to a subject with elevated blood pressure.

Another aspect provides a method for modulating IL-1 receptor activation in a subject with elevated blood pressure comprising, consisting of, or consisting essentially of administering an IL-1 receptor antagonist.

Another aspect of the present disclosure provides a method of modifying IL-1 receptor signaling to treat hypertension comprising, consisting or, or consisting essentially of administering an IL-1 receptor antagonist.

Yet another aspect of the present disclosure provides a composition comprising an IL-1 receptor antagonist that decreased blood pressure to a therapeutically effective level in a subject with hypertension.

### BRIEF DESCRIPTION OF THE DRAWINGS

FIG. 1 is graph depicting mean arterial pressure of uni-nephrectomized IL-1 R-deficient (IL-1R KO) and wild-type (WT) mice with chronically infused with angiotensin II (Ang II) for 4 weeks.

FIG. 2 is graph illustrates IL-1 receptor stimulation augments renal sodium reabsorption during RAS-mediated hypertension.

FIGS. 3A and 3B are graphs illustrating the mean arterial pressure after treatments with Anakinra (50 mg/kg/d IP) to wild-type mice in AngII-dependent model over 2 weeks (3A) and 3 weeks (3B).

FIG. 4 is graph depicting baseline blood pressures after uni-nephrectomy measured by radiotelemetry and found to be similar in the BMWT (wild-type control) and BMKO (AT₁ receptor-deficient bone marrow chimeras) (116±3 vs. 117±1 mm Hg).

FIG. 5 shows enhanced accumulation of F4/80+ macrophages in IL-1R1 KO kidneys following 4 weeks of chronic Ang II infusion.

FIGS. 6A and 6B shows renal expression of eNOS and iNOS mRNA compared to wild type controls in Ang II-infused IL-1R KO mice.

FIG. 7 shows sodium balance in Ang II-infused IL-1R KO mice. During the second week of Ang II, the WT mice remained in positive sodium balance whereas IL1 receptor-KO mice switched to a negative sodium balance (208.84±44.84 vs. −100.66±100.74 μmol/6 days, p=0.013).

FIG. 8 shows the response to Furosemide (FURO) in Ang II-infused wild type and IL-1R KO mice.

FIG. 9A shows the effect on cardiac hypertrophy and FIG. 9B shows the effect on albuminuria after treatment with IL-1 receptor antagonist anakinra.

### DETAILED DESCRIPTION OF THE INVENTION

Before the disclosed methods and materials are described, it is to be understood that the aspects described herein are not limited to specific embodiments, apparati, or configurations, and as such can, of course, vary. It is also to be understood that the terminology used herein is for the purpose of describing particular aspects only and, unless specifically defined herein, is not intended to be limiting.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

As used herein the term “contacting” includes the physical contact of at least one substance to another substance.

As used herein the term “hypertension” means high blood pressure, i.e., a condition present when blood flows through the blood vessels with a force greater than normal. The hypertension of the disclosure may be primary or secondary. Primary hypertension may develop gradually over time (e.g., several years). Secondary hypertension may be caused by an underlying condition, such as kidney problems, obstructive sleep apnea, adrenal gland tumors, thyroid problems, alcohol or drug abuse, certain medication, or certain congenital disorders. In the methods of the disclosure, hypertension includes blood pressure as those provided by Joint National Committee on Prevention, Detection, Evaluation, and Treatment of High Blood Pressure (Chobanian et al. “Seventh report of the Joint National Committee on Prevention, Detection, Evaluation, and Treatment of High Blood Pressure.” Hypertension; 42: 1206-1252 (2003)). For example, the hypertension includes blood pressure in adults over 140 mmHg systolic and over 90 mmHg diastolic. In certain embodiments, hypertension in adults includes blood pressure over 160 mmHg systolic and over 100 mmHg diastolic. In certain embodiments, hypertension includes prehypertension where the blood pressure in adults is between about 120 and 139 mmHg systolic and between about 80 and 89 mmHg diastolic.

As used here, the terms “treatment” and “treating” means:

(i) inhibiting the progression the disease;

(ii) prophylactic use for example, preventing or limiting development of a disease, condition or disorder in an individual who may be predisposed or otherwise at risk to the disease, condition or disorder but does not yet experience or display the pathology or symptomatology of the disease;

(iii) inhibiting the disease; for example, inhibiting a disease, condition or disorder in an individual who is experiencing or displaying the pathology or symptomatology of the disease, condition or disorder;

(iv) ameliorating the referenced disease state, for example, ameliorating a disease, condition or disorder in an individual who is experiencing or displaying the pathology or symptomatology of the disease, condition or disorder (i.e., reversing or improving the pathology and/or symptomatology) such as decreasing the severity of disease; or

(v) eliciting the referenced biological effect.

As used herein, the terms “individual,” “patient,” or “subject” are used interchangeably, refers to any animal, including mammals, preferably mice, rats, other rodents, rabbits, dogs, cats, swine, cattle, sheep, horses, or primates, and most preferably humans. In certain embodiments, the “subject” is human.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the phrase “therapeutically effective level” refers to the biological or medicinal response level that is being sought in a tissue, system, animal, individual or human by a researcher, veterinarian, medical doctor or other clinician.

In view of the present disclosure, the methods and active materials described herein can be configured by the person of ordinary skill in the art to meet the desired need. In general, the disclosed materials, methods, and apparati provide improvements in treatment or amelioration of hypertension.

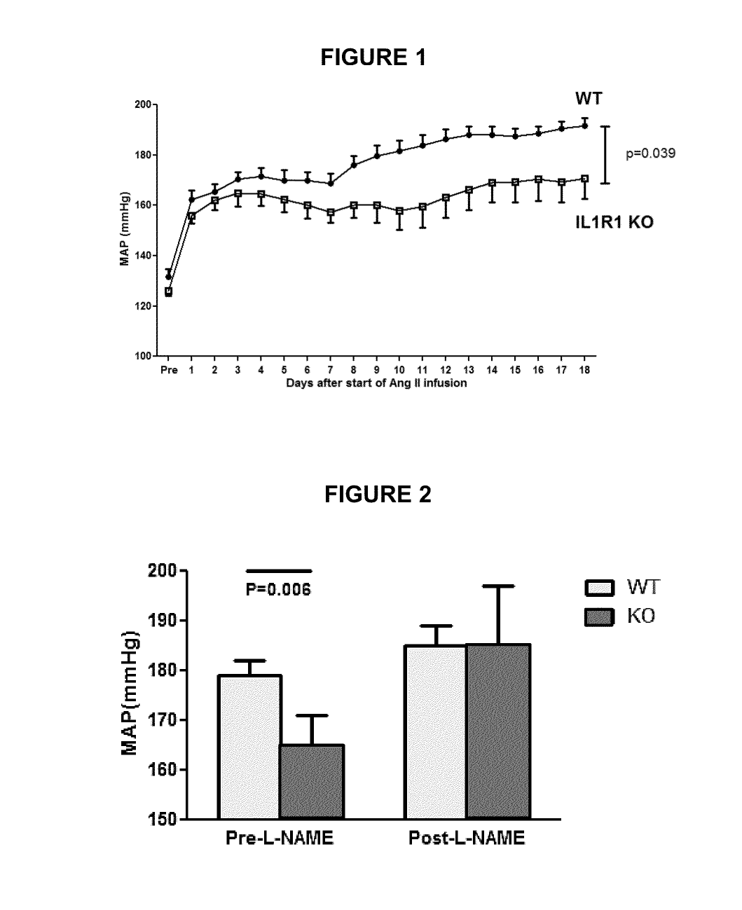

To study the role of IL-1 receptor (IL-1 R) activation in hypertension, uni-nephrectomized IL-1 R-deficient(IL-1R KO) and wild-type (WT) mice was chronically infused with angiotensin II (Ang II; 1000 ng/kg/min) for 4 weeks using an implanted osmotic mini-pump after measurement of baseline blood pressures. Chronic angiotensin II infusion in rodents is a widely accepted model of hypertension that leads to the same complications in rodents as those seen in humans including cardiac hypertrophy and kidney damage. Unilateral nephrectomy renders the remaining kidney more susceptible to injury in this hypertension model. In the experiments of the disclosure baseline mean arterial pressures (MAPs) were similar between groups. By contrast, after the first week of Ang II, IL-1 R KOs had markedly blunted MAP elevations vs. WTs (163±6 vs. 179±3 mm Hg; p<0.04), indicating that IL-1 receptor activation potentiates blood pressure elevation (FIG. 1.)

Consistent with their lower blood pressures, IL-1 R KO mice showed evidence of less target organ damage following 4 weeks of Ang II infusion. For example, the Ang 11-infused IL-1 R KO mice had less cardiac hypertrophy than WJ mice as measured by heart weight/body weight ratios (7.5±0.9 vs. 9.3±0.8 mg/g, p<0.001). Loss of albumin in the urine is a marker for kidney glomerular damage in humans, and Ang 11-infused IL-1 R KO mice had nearly 40% less in albuminuria than Ang 11-infused WT animals (5.90±0.93 vs. 9.38±1.43 mg/mg Cr; p=0.05). Similarly, renal mRNA expression of the kidney injury marker neutrophil gelatinase-associated lipocalin was dramatically reduced in Ang 11-infused IL-1R KO animals compared to WT controls (0.37±0.06 vs. 1±0.19 au; p=0.008). Thus, the lower blood pressures in IL-1 receptor-deficient mice led to reduced hypertensive damage to the heart and kidney.

Because IL-1 is secreted by macrophages, macrophage infiltration into the kidneys of the experimental animals was examined. After 4 weeks of hypertension, IL-1R KO kidneys contained more F4/80+ macrophages than WTs (18±1 vs. 14±2 per HPF; p=0.03). Among these macrophages, monocytic myeloid-derived suppressor cells (Mo-MDSC) that produce nitric oxide (NO) showed 60% greater accumulation in IL-1 R KO kidneys than in WTs by FACS (p=0.05). In turn, the KO group had enhanced renal mRNA expression of the NO-generating enzyme iNOS (1.7±0.3 vs. 1.0±0.2 au; p<0.05) and higher excretion of NO metabolites vs. WTs (152±46 vs. 60±15 nmol/mg Cr; p=0.05). NO inhibits renal sodium (Na) reabsorption, and during the 2″d wk of Ang II infusion when WT and KO BPs separated, only the KOs entered negative Na balance (−16.8±16.8 vs. WT 34.8±7.5 1-Jmol/day, p=0.01). To confirm that preserved NO bioavailability in the Ang 11-infused KOs led to their lower BP vs. WTs, we blocked NO generation in the groups with L-NAME starting on day 7 of Ang II when the WT and KO BPs were 179±3 vs. 165±6 mm Hg (p<0.01). By day 14, BPs converged (186±5 vs. 185±8; p=NS), (FIG. 2.) As NO is known to inhibit the NKCC2 sodium transporter in the kidney, NKCC2 activity at day 10 of Ang II was measured by quantitating urine sodium to creatinine ratios (UNa/UCr) 3 hours after an IP injection of saline with or without furosemide. After saline alone, the KOs had a higher UNa/UCr than the WTs (237±38 vs. 120±14 mmollmmol; p=0.01) whereas the UNa/UCr converged in response to furosemide (481±1 03 vs. 453±60; p=NS). Thus, reduced NKCC2 activity in the IL-1 R KOs protects them from Ang 11-induced sodium retention.

Anakinra (Kineret®) is a recombinant, nonglycosylated form of the human interleukin-1 receptor antagonist (IL-1Ra). Anakinra differs from native human IL-1Ra in that it has the addition of a single methionine residue at its amino terminus. Anakinra consists of 153 amino acids and has a molecular weight of 17.3 kilodaltons. It is produced by recombinant DNA technology using an E coli bacterial expression system and available from SOBI, Inc. (Swedish Orphan Biovitrum AB (publ), Stockholm, Sweden). It has been approved for the treatment of rheumatologic diseases including rheumatoid arthritis, and the treatment of Neonatal-Onset Multisystem Inflammatory Disease (NOMID).

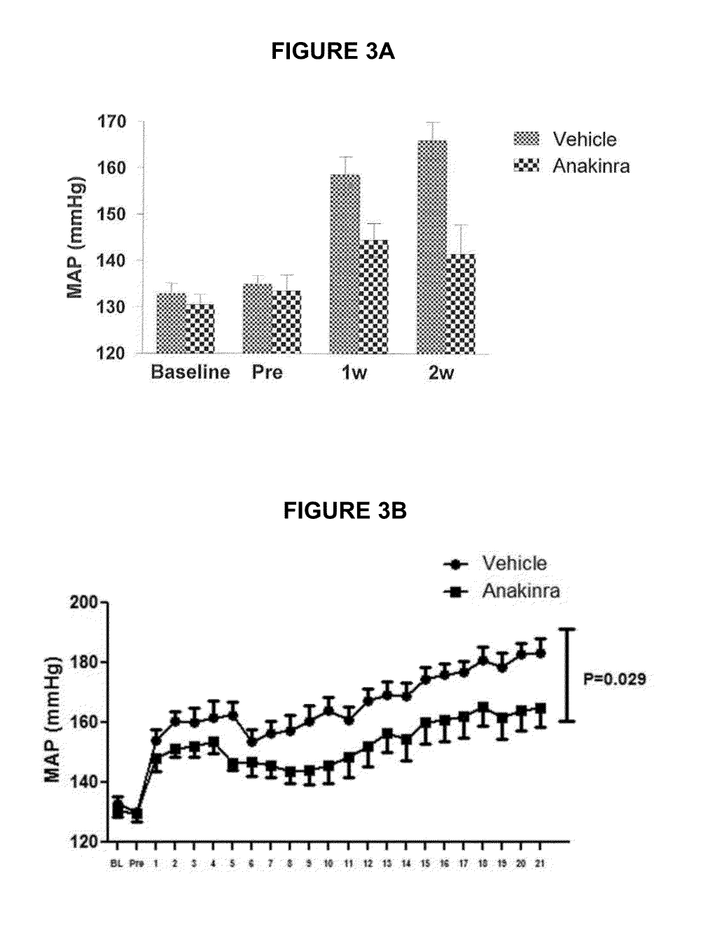

In the present disclosure, Anakinra was administered at 50 mg/kg/day IP to wild-type mice in AngII-dependent hypertension model. FIG. 3A shows that that through weeks 1 and 2 of AngII-dependent hypertension (“1w” and “2w” in FIG. 3A), the ability of the IL-1 receptor antagonist to limit blood pressure elevation is as robust as genetic deletion of the receptor, despite no impact on baseline blood pressure (“Pre”).

FIG. 3B illustrates the effect of treatment with 50 mg/kg/day IP once daily beginning 3 days prior to and continuing throughout the Ang II infusion period. Anakinra had no effect on baseline blood pressures, consistent with the lack of hypotension in clinical trials in patients with rheumatic diseases. However, following the initiation of Ang II infusion, the protection from hypertension with anakinra was as profound as that seen in the IL-1R KO mice, highlighting the translational potential of this anti-hypertensive therapy.

Therefore, in one aspect, the disclosure provides methods for treating or ameliorating hypertension comprising, consisting of, or consisting essentially of, administering an IL-1 receptor antagonist at a therapeutically effective level to a subject with elevated blood pressure.

In one embodiment, the treatment or amelioration of hypertension comprises decreasing blood pressure to a therapeutically effective level. In one embodiment, the blood pressure (e.g., systolic blood pressure) is decreased by at least about 5 mm Hg, or at least about 7 mm Hg, or at least about 7.5 mm Hg, or at least about 8 mm Hg, or at least about 9 mm Hg, or at least about 10 mm Hg, or at least about 11 mm Hg, or at least about 12 mm Hg, or at least about 13 mm Hg, or at least about 14 mm Hg, or at least about 15 mm Hg, or at least about 16 mm Hg, or at least about 17 mm Hg, or at least about 18 mm Hg, or at least about 19 mm Hg, or at least about 20 mm Hg, or more than about 5 mm Hg, or more than about 10 mm Hg, or more than about 15 mm Hg, or more than about 16 mm Hg, or more than about 17 mm Hg, or more than about 18 mm Hg, or more than about 19 mm Hg, or more than about 20 mm Hg, or more than about 25 mm Hg from the elevated blood pressure level for the subject.

In one embodiment, the treatment or amelioration of hypertension comprises decreasing blood pressure to less than about 120/80 mm Hg systolic/diastolic. In certain embodiments, the decreasing blood pressure is to less than about 140/90 mm Hg systolic/diastolic. In other embodiments, the treatment or amelioration of hypertension comprises treating Stage 2 hypertension (more than 160/100 mm Hg systolic/diastolic) wherein the blood pressure is decreased to Stage 1 levels (140-159/90-99 mm Hg systolic/diastolic) or less.

In one embodiment, the blood pressure (e.g., systolic blood pressure) is decreased by at least about 2% as compared to the elevated blood pressure level for the subject, or at least about 3%, or at least about 4%, or at least about 5%, or at least about 6%, or at least about 7%, or at least about 8%, or at least about 9%, or at least about 10%, or at least about 11%, or at least about 12%, or at least about 13%, or at least about 14%, or at least about 15%, or at least about 17%, or at least about 19%, or at least about 20%, or at least about 22%, or at least about 25%, or more than about 2%, or more than about 3%, or more than about 4%, or more than about 5%, or more than about 8%, or more than about 10%, or more than about 11%, or more than about 12%, or more than about 15%, or more than about 16%, or more than about 17%, or more than about 18%, or more than about 19%, or more than about 20%, or more than about 25% as compared to the elevated blood pressure level for the subject.

In one embodiment, the IL-1 receptor antagonist is Anakinra

Anakinra may be administered in a therapeutically effective amount. Thus, in certain embodiments, Anakinra is administered at about 0.01 to about 500 mg/kg daily dose. In other embodiments, Anakinra is administered in a daily dose of about 0.01 to about 400 mg/kg, or about 0.01 to about 300, or about 0.01 to about 200, or about 0.01 to about 150, or about 0.01 to about 100, or about 0.01 to about 75, or about 0.01 to about 50, or about 1 to about 500, or about 1 to about 400, or about 1 to about 300, or about 1 to about 200, or about 1 to about 150, or about 1 to about 100, or about 1 to about 75, or about 1 to about 50, or about 10 to about 500, or about 10 to about 400, or about 10 to about 300, or about 10 to about 200, or about 10 to about 150, or about 10 to about 100, or about 10 to about 75, or about 10 to about 50, or about 30 to about 500, or about 30 to about 400, or about 30 to about 300, or about 30 to about 200, or about 30 to about 150, or about 30 to about 100, or about 30 to about 75, or about 30 to about 50 mg/kg. In other embodiments, Anakinra is administered in a daily dose of about 20 to about 100 mg/kg, or about 20 to about 80, or about 20 to about 70, or about 20 to about 60, or about 30 to about 80, or about 30 to about 75, or about 30 to about 70, or about 30 to about 60, or about 40 to about 100, or about 40 to about 80, or about 40 to about 70, or about 40 to about 60 mg/kg, or about 50 mg/kg. In other embodiments, Anakinra is administered in a daily dose of about 50 to about 200 mg, or about 50 to about 150 mg, or about 80 to about 120 mg.

In one embodiment, the IL-1 receptor antagonist is Canakinumab (ACZ885, ILARIS®). Canakinumab is a human anti-IL-1β monoclonal antibody currently available from Novartis Pharmaceuticals Corp., East Hanover, N.J.

In one embodiment, the IL-1 receptor antagonist is administered together with one or more therapeutic agents. The agent(s) can be combined with the IL-1 receptor antagonist in a single dosage form, or the agents can be administered simultaneously or sequentially as separate dosage forms. In another embodiment, the one or more therapeutic agents are angiotensin-converting-enzyme inhibitors (ACE inhibitors). For example, ACE inhibitors include, but are not limited to, captopril, zofenopril, enalapril, ramipril, quinapril, perindopril, lisinopril, benazepril, imidapril, trandolapril, cilazapril, moexipril, and fosinopril. In another embodiment, the one or more therapeutic agents are angiotensin II receptor antagonists (also known as angiotensin receptor blockers (ARBs)). For example, ARBs include, but are not limited to, valsartan, telmisartan, losartan, irbesartan, azilsartan, olmesartan, losartan, fimasartan, eprosartan, and candesartan.

Another aspect provides a method for modulating IL-1 receptor activation in a subject with elevated blood pressure comprising, consisting of, or consisting essentially of administering an IL-1 receptor antagonist.

Another aspect of the present disclosure provides a method of modifying IL-1 receptor signaling to treat hypertension comprising, consisting or, or consisting essentially of administering an IL-1 receptor antagonist.

In one embodiment, modulating IL-1 receptor activation and/or modifying IL-1 receptor signaling and/or comprises decreasing blood pressure (e.g., systolic blood pressure) to a therapeutically effective level. In one embodiment, the blood pressure is decreased by at least about 5 mm Hg, or at least about 7 mm Hg, or at least about 7.5 mm Hg, or at least about 8 mm Hg, or at least about 9 mm Hg, or at least about 10 mm Hg, or at least about 11 mm Hg, or at least about 12 mm Hg, or at least about 13 mm Hg, or at least about 14 mm Hg, or at least about 15 mm Hg, or at least about 16 mm Hg, or at least about 17 mm Hg, or at least about 18 mm Hg, or at least about 19 mm Hg, or at least about 20 mm Hg, or more than about 5 mm Hg, or more than about 10 mm Hg, or more than about 15 mm Hg, or more than about 16 mm Hg, or more than about 17 mm Hg, or more than about 18 mm Hg, or more than about 19 mm Hg, or more than about 20 mm Hg, or more than about 25 mm Hg from the elevated blood pressure level for the subject. In certain embodiments, decreasing blood pressure to a therapeutically effective level is decreasing the blood pressure to less than about 120/80 mm Hg systolic/diastolic. In certain embodiments, decreasing blood pressure to a therapeutically effective level is decreasing the blood pressure to less than about 140/90 mm Hg systolic/diastolic. In one embodiment, the decreasing blood pressure is to less than about 120/80 mm Hg systolic/diastolic. In certain embodiments, the decreasing blood pressure is to less than about 140/90 mm Hg systolic/diastolic. In other embodiments, decreasing the blood pressure is wherein Stage 2 hypertension (more than 160/100 mm Hg systolic/diastolic) is decreased to Stage 1 levels (140-159/90-99 mm Hg systolic/diastolic) or less.

In one embodiment, the blood pressure (e.g., systolic blood pressure) is decreased by at least about 2% as compared to the elevated blood pressure level for the subject, or at least about 3%, or at least about 4%, or at least about 5%, or at least about 6%, or at least about 7%, or at least about 8%, or at least about 9%, or at least about 10%, or at least about 11%, or at least about 12%, or at least about 13%, or at least about 14%, or at least about 15%, or at least about 17%, or at least about 19%, or at least about 20%, or at least about 22%, or at least about 25%, or more than about 2%, or more than about 3%, or more than about 4%, or more than about 5%, or more than about 8%, or more than about 10%, or more than about 11%, or more than about 12%, or more than about 15%, or more than about 16%, or more than about 17%, or more than about 18%, or more than about 19%, or more than about 20%, or more than about 25% as compared to the elevated blood pressure level for the subject.

In one embodiment, modulating IL-1 receptor activation and/or modifying IL-1 receptor signaling and/or comprises administering an IL-1 receptor antagonist that is Anakinra Anakinra may be administered in a therapeutically effective amount. Thus, in certain embodiments, Anakinra is administered at about 0.01 to about 500 mg/kg daily dose. In other embodiments, Anakinra is administered in a daily dose of about 0.01 to about 400 mg/kg, or about 0.01 to about 300, or about 0.01 to about 200, or about 0.01 to about 150, or about 0.01 to about 100, or about 0.01 to about 75, or about 0.01 to about 50, or about 1 to about 500, or about 1 to about 400, or about 1 to about 300, or about 1 to about 200, or about 1 to about 150, or about 1 to about 100, or about 1 to about 75, or about 1 to about 50, or about 10 to about 500, or about 10 to about 400, or about 10 to about 300, or about 10 to about 200, or about 10 to about 150, or about 10 to about 100, or about 10 to about 75, or about 10 to about 50, or about 30 to about 500, or about 30 to about 400, or about 30 to about 300, or about 30 to about 200, or about 30 to about 150, or about 30 to about 100, or about 30 to about 75, or about 30 to about 50 mg/kg. In other embodiments, Anakinra is administered in a daily dose of about 20 to about 100 mg/kg, or about 20 to about 80, or about 20 to about 70, or about 20 to about 60, or about 30 to about 80, or about 30 to about 75, or about 30 to about 70, or about 30 to about 60, or about 40 to about 100, or about 40 to about 80, or about 40 to about 70, or about 40 to about 60 mg/kg, or about 50 mg/kg.

In some embodiments, the method comprises the administration of the IL-1 receptor antagonist in a pharmaceutical composition having at least one pharmaceutically acceptable carrier, solvent, adjuvant or diluent.

The materials described herein may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. The pharmaceutical compositions described herein may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compositions disclosed herein may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

The compositions disclosed herein may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The materials of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the material in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active material in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The materials may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Dosage levels of the order of mg per kilogram of body weight per day (as described above) are useful in the treatment of the above-indicated conditions. The amount of active ingredient that may be combined with the carrier materials to produce a single dosage form will vary depending upon the host treated and the particular mode of administration. Dosage unit forms will generally contain between from about 0.01 mg to about 500 mg of an active ingredient. The daily dose can be administered in one to four doses per day. In the case of skin conditions, it may be preferable to apply a topical preparation of materials of this invention to the affected area two to four times a day.

It will be understood, however, that the specific dose level for any particular patient will depend upon a variety of factors including the activity of the specific material employed, the age, body weight, general health, sex, diet, time of administration, route of administration, and rate of excretion, drug combination and the severity of the particular disease undergoing therapy.

The materials and methods of the disclosure are illustrated further by the following examples, which are not to be construed as limiting the disclosure in scope or spirit to the specific procedures and in them.

### Examples

### Example 1

### AT₁ Receptors on Bone Marrow-Derived Cells Regulate Ang 11-Dependent Hypertension

As T lymphocytes and macrophages both infiltrate the kidney during angiotensin (Ang) II infusion, and both of these cell lineages are derived from bone marrow progenitors, it was hypothesized that activation of type 1 (AT₁) receptors on bone marrow-derived cells might play a role in the pathogenesis of Ang II-dependent hypertension and its complications. To test this hypothesis, the inventors eliminated AT₁ receptor-mediated responses from bone marrow-derived cells by generating AT₁ receptor-deficient bone marrow chimeras (BMKO) and wild-type controls (BMWT). To this end, 129/SvEv mice were lethally irradiated and transplanted the same day with bone marrow from syngeneic 129/SvEv donors that were either wild-type or deficient in the dominant murine AT₁ receptor isoform, AT_{1A}. At the conclusion of the experiment 14 weeks later, Real-time PCR for the Agtr1a gene was performed starting with splenocyte RNA to confirm bone marrow engraftment in the recipients. Compared to recipients of wild-type bone marrow (BMWT), recipients of AT_{1A} receptor-deficient bone marrow (BMKO) had a 98% reduction in AT_{1A} receptor expression [0.02±0.00 vs. 1.00±0.07 arbitrary units (au); p<0.00001], thus confirming deletion of the AT_{1A} receptor from immune cells in these animals. AT_{1A} receptor deletion was also confirmed by PCR analysis of genomic DNA in the recipient splenocytes. At the DNA level, Agtr1a expression could not be detected in the splenocytes of the BMKO group (Agtr1a^{+/+} allele=700 bp, Agtr1a^{-/-} allele=800 bp).

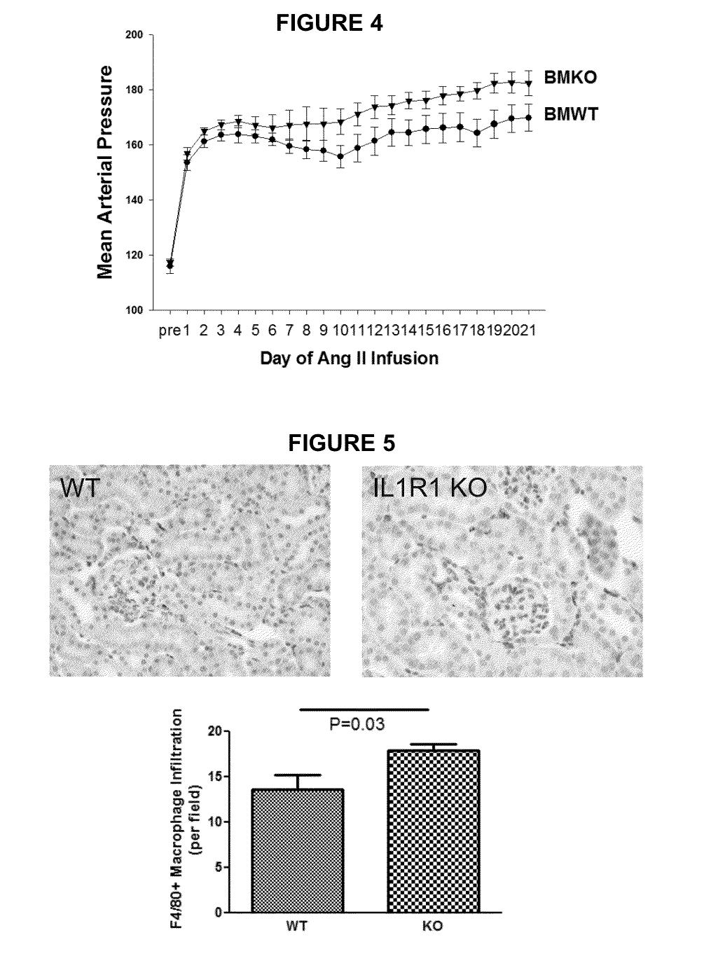

Following bone marrow transfer, 8 weeks for immune reconstitution were allowed and then performed unilateral nephrectomy to enhance salt-sensitivity. After uni-nephrectomy, baseline blood pressures were measured by radiotelemetry in the experimental groups. As illustrated in FIG. 4, baseline mean blood pressures in the BMWT group (116±3 mm Hg) were quite similar to those previously measured in non-transplanted wild-type mice, indicating that the bone marrow transfer did not significantly impact baseline blood pressures. Moreover, baseline blood pressures in the BMWT and BMKO groups (116±3 vs. 117±1 mm Hg; p=NS) were virtually identical suggesting that AT₁ receptors on bone marrow-derived cells do not make a unique contribution to the maintenance of baseline blood pressure.

After measurement of baseline blood pressures, an osmotic mini-pump was implanted subcutaneously to chronically infuse angiotensin II (Ang II; 1000 ng/kg/min) for 4 weeks. Following initiation of Ang II infusion, blood pressures in the BMWT group rose to nearly 160 mm Hg and remained elevated throughout the infusion period (FIG. 4) in a pattern similar to that previously seen in wild-type animals infused with Ang II. These data suggest that the bone marrow transfer procedure did not significantly alter the chronic hypertensive response to Ang II. Surprisingly, during Ang II infusion, the BMKO group had higher blood pressures than the BMWT group (FIG. 4), indicating a protective effect of AT₁ receptors on bone marrow-derived cells with regard to blood pressure elevation. The difference in blood pressure between the groups was particularly evident in the second and third weeks of the Ang II infusion period during which the increase in blood pressure over baseline was more than 20% greater in the BMKO group (+58±3 mm Hg) than in the BMWT controls (+47±3 mm Hg; p=0.03). Thus, a therapy that could preserve the beneficial effects of AT₁ receptors on immune cells to suppress blood pressure elevation in patients treated with angiotensin receptor blockers (ARBs) enhances blood pressure control in patients with resistant hypertension

### Example 2

### AT₁ Receptors on Bone Marrow-Derived Cells Influence Renal Generation of Macrophage Cytokines in Ang 11-Induced Hypertension

Following 4 weeks of Ang II-dependent hypertension, the BMWT and BMKO cohorts both showed robust macrophage accumulation in the renal interstitium. Macrophages infiltrating the kidney can directly secrete cytokines and also regulate secretion of cytokines from renal parenchymal cells through paracrine mechanisms. These cytokines, in turn, can have important effects on blood pressure regulation. Therefore, Real-time PCR was used following 4 weeks of Ang II to examine renal expression of several macrophage cytokines that have been shown to affect blood pressure or kidney injury including tumor necrosis factor-a (TNF-a), interleukin-1a (IL-1a), and interleukin-1b (IL-1b). TNF-a was numerically but not significantly upregulated in the Ang II-infused BMKO mice (data not shown). By contrast, IL-1a and IL-1b were significantly upregulated in the kidneys from the Ang II-infused BMKO mice. Moreover, activation of the IL-1 receptor triggers release of the pro-hypertensive cytokine IL-6, and expression of IL-6 was also enhanced in the kidneys of the Ang II-infused BMKO mice compared to BMWT controls. Without beoing bound by a particular theory, it was believed that exaggerated IL-1 generation in the BMKO group led to their enhanced hypertensive response.

To examine the contribution of both IL-1 isoforms to Ang II-dependent hypertension, 129/SvEv wild-type (WT) and IL-1 receptor-deficient (IL-1R KO) mice were subjected to Ang II-induced hypertension model as described above (n>10 per group). Both groups had similar blood pressures at baseline. However, the IL-1R KO mice were markedly protected from Ang II-induced blood pressure elevation (FIG. 1). Consistent with their lower pressures, the IL-1R KOs had significantly less cardiac hypertrophy than WT controls following 4 weeks of hypertension as measured by heart to body weight ratios (7.5±0.9 vs. 9.3±0.8 mg/gm; p<0.001). Thus, IL-1 receptor stimulation augments the degree of hypertension induced by activation of the renin angiotensin system (RAS) leading to exaggerated cardiovascular damage.

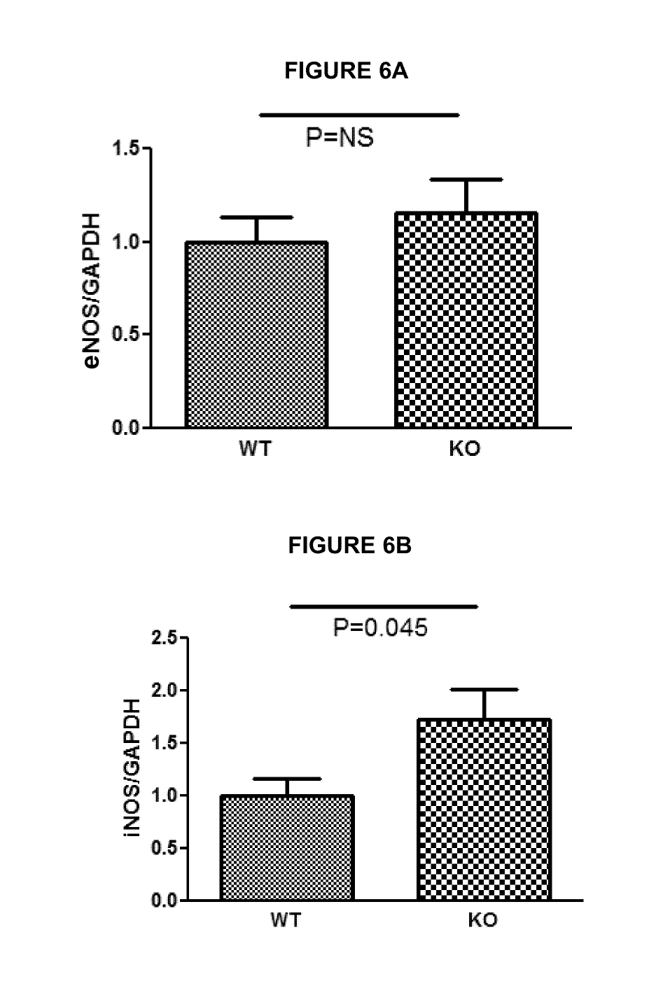

As IL-1 is secreted primarily by macrophages, the number of F4/80-positive macrophages infiltrating the kidney were examined after 4 weeks of Ang II in the WT and IL-1R KO groups. It was found 32% more rather than fewer macrophages in the IL-1R KO kidneys compared to WTs (17.9±0.8 vs. 13.6±1.7; p=0.03; FIG. 5). One macrophage subset, the myeloid-derived suppressor cell, or MDSC, produces nitric oxide (NO), which promotes vasodilation and sodium excretion was found to be suppressing hypertensive response. A fluorescent cell sorting strategy was used to characterize the subsets of macrophages infiltrating the kidney at day 7 of Ang II infusion when the blood pressures began to separate between groups. At that timepoint, the kidney was harvested, prepared single cell suspensions, and double-labeled the cells for CD45 and CD11b to mark infiltrating macrophages. These macrophages were then stratified based on their levels of Ly6C and Ly6G staining that together identify subpopulations of myeloid-derived suppressor cells or MDSC. The monocytic-MDSC subset that produces nitric oxide and expresses Ly6C but not Ly6G was the major subset of MDSCs in the kidneys from Ang II-infused animals. Moreover, it was noted a distinct shift away from the Ly6C and G double-positive “immature” myeloid cells and towards the Ly6C single-positive monocytic, NO-producing MDSC in the kidneys from IL-1R KO group. Indeed, in the IL-1R KO kidneys, there was a 60% increase in monocytic MDSCs (17.5±3.0 vs. 11.1±1.5%; p=0.05) and a 50% decrease in immature myeloid cells (10.6±1.7 vs. 20.7±3.7%, p=0.049). The enzyme responsible for catalyzing the production of NO in the monocytic MDSC is inducible NOS (iNOS=NOS2). Consistent with enhanced infiltration of iNOS-producing MDSC in the KO kidneys, markedly exaggerated renal expression of iNOS mRNA compared to WT controls (1.73±0.29 vs. 1.00±0.16, p=0.045) (FIG. 6B) was found. In turn, as a marker of local NO production, 24-hour urinary excretion of NO metabolites was increased more than 2-fold in the IL-1 receptor KO group at day 7 of Ang II (153±46 vs. 60±15 nmol/24 h; p<0.05). Without being bound by a particular theory, it is believe that these data suggest that IL-1 receptor activation potentiates Ang II-induced blood pressure elevation by suppressing the accumulation of NO-producing MDSC in the kidney early in the course of RAS-mediated hypertension. Such an immune-mediated mechanism is not targeted by any anti-hypertensive therapy currently in use.

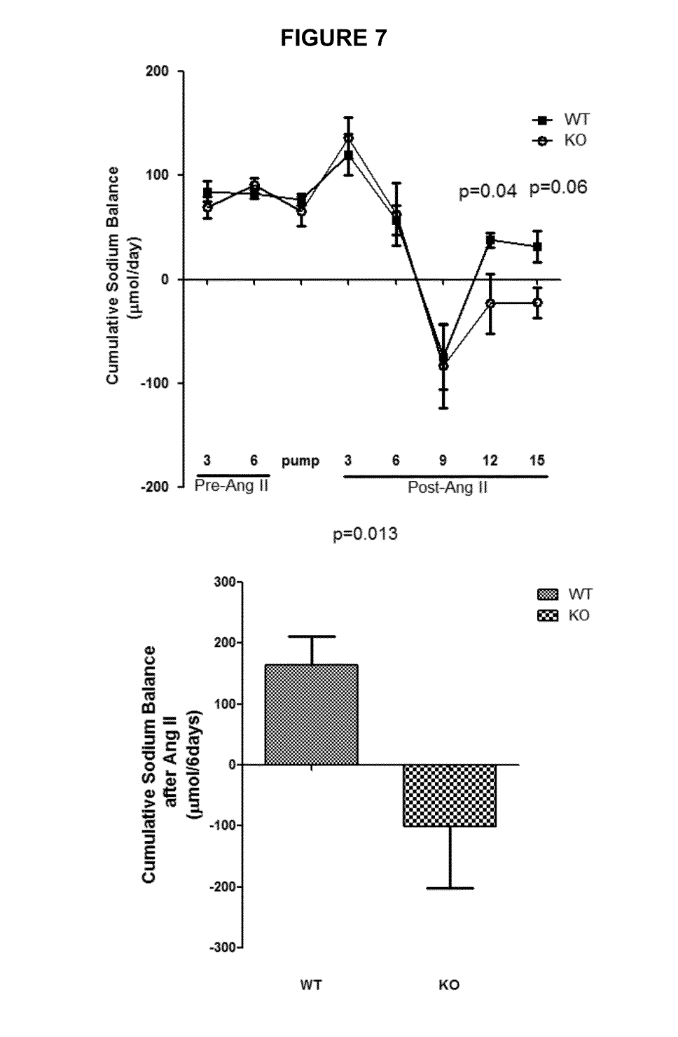

Next, WT and IL-1R KO mice were placed into metabolic cages 1 week before and 2 weeks following the initiation of chronic Ang II infusion and fed them a gel food to standardize sodium intake. For each day, sodium ingestion and excretion was quantitated to compute a net sodium balance (FIG. 7). Prior to Ang II infusion, the net sodium balance was similar between the 2 groups. However, concurrent with the separation of blood pressures during 2nd of week of Ang II, the WT mice remained in positive sodium balance whereas IL1 receptor-KO mice switched to a negative sodium balance (209±45 vs. −101±101 μmol/6 days, p=0.013). These data indicate that IL-1 receptor activation potentiates Ang II-induced sodium retention, which could underlie the blood pressure differences seen in the experimental groups.

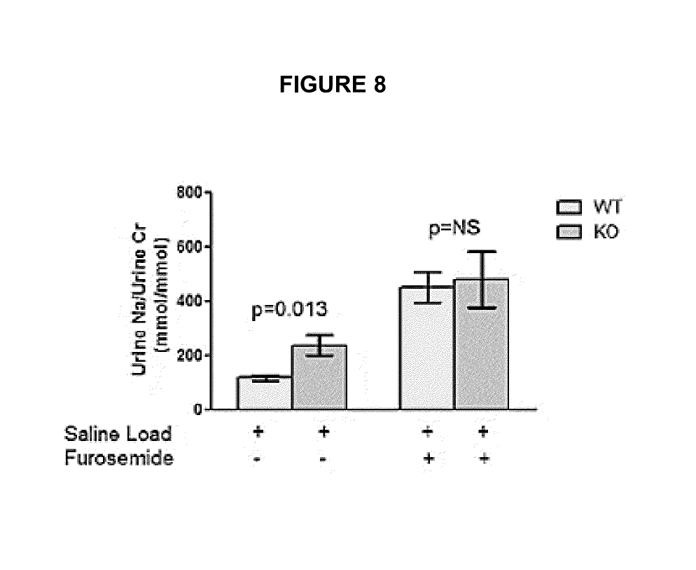

Therefore, to examine NKCC2 activity in the groups at day 10 of Ang II when the differences in sodium excretion was noted, 3-hour sodium excretion was quantitated in response to an acute intraperitoneal (IP) furosemide challenge (FIG. 8). In response to IP saline injection alone, the IL-1RKO mice had exaggerated urine sodium to creatinine ratios, consistent with their enhanced natriuresis in original balance study (237±38 vs. 120±9%; p=0.016). However blockade of NKCC2 with IP furosemide abrogated this difference (481±103 vs. 453±56%; p=NS), suggesting that impaired NKCC2 activity in the Ang II-infused IL-1R KO mice accounts for their preserved capacity to excrete sodium during RAS-mediated hypertension. Without being bound by a particular theory, it is believed that IL-1 receptor activation stimulates renal sodium reabsorption via NKCC2-mediated sodium transport during Ang II-dependent HTN.

### Example 3

### Pharmacologic Deficiency of the IL-1 Mitigates Blood Pressure Elevation

Uni-nephrectomized IL-1 R-deficient(IL-1R KO) and wild-type (WT) mice was chronically infused with (1000 ng/kg/min) for 4 weeks using an implanted osmotic mini-pump after measurement of baseline blood pressures. L-Nitroarginine Methyl Ester (L-NAME) was administered in the drinking water starting at day 7 of Ang II infusion (FIG. 2). At day 7 of Ang II prior to L-NAME administration, the IL-1R KO mice (KO) had significantly lower BPs than the WTs as seen in our original hypertension experiment (165±6 vs. 179±3 mm Hg; p<0.01). By contrast, following L-NAME treatment, blood pressures in the two groups converged (185±8 vs. 186±5; p=NS). Without being bound by a particular theory, it is believed that deprivation of NO availability abrogated the protection from Ang II-induced blood pressure elevation in IL-1R KO mice.

### Example 4

### Anakinra Blockade of the IL-1 Receptor Dramatically Attenuates Hypertension in Mice

Uni-nephrectomized IL-1 R-deficient(IL-1R KO) and wild-type (WT) mice was chronically infused with (1000 ng/kg/min) for 4 weeks using an implanted osmotic mini-pump after measurement of baseline blood pressures. The mice were then treated with vehicle or anakinra (50 mg/kg/day IP) once daily beginning 3 days prior to and continuing throughout the Ang II infusion period (FIG. 3). Blood pressure was monitored continuously using radiotelemetry. Anakinra had no effect on baseline blood pressures, consistent with the lack of hypotension in clinical trials in patients with rheumatic diseases; however, following the initiation of Ang II infusion, the protection from hypertension with anakinra was as profound as that seen in the IL-1R KO mice.

### Example 5

### Anakinra Blockade of the IL-1 Receptor Limits Target Organ Damage in Hypertension in Mice

Consistent with the blunted hypertensive response in the Ang II-infused IL-1 receptor-KOs, these mice also had significantly less cardiac hypertrophy versus WTs as measured by heart to body weight ratios at both 14 and 28 days of Ang II infusion: 6.72±0.28 vs. 7.41±0.18, p=0.058 n=7, at day 14; 7.49±0.86 vs. 9.25±0.84, p<0.001, at day 28.

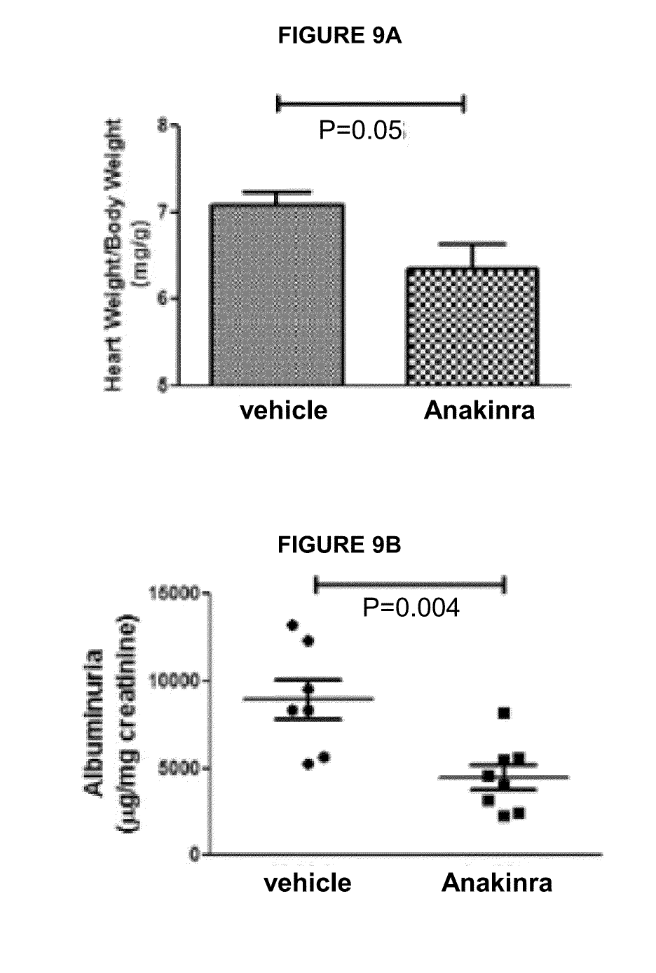

In the mice treated with IL-1 receptor antagonist anakinra, the decrease in cardiac hypertrophy with anakinra was as profound as that seen in the IL-1R KO mice (FIG. 9A).

Next, levels of albuminuria, which is a marker for kidney damage, were measured in Ang II-infused IL-1R Kos and wild type mice. It was observed that the levels of albuminuria were reduced by 37% in the IL1R KO mice vs. the wild type: 5899±929 vs. 9380±1423, n=9,10, p=0.052 (37.1% reduction); for 24 hour albuminuria, 3574 vs. 5082, p=0.0810 (30.4% reduction). Moreover, renal expression for NGAL, another marker for kidney injury, showed a 63% reduction in the KO mice compared to controls (0.37±0.06 vs. 1±0.19, n=8, p=0.008). Collectively, these data suggest activation of IL-1 R contributes to Ang II-induced kidney injury.

Similarly, in the mice treated with anakinra, the decrease in albuminuria was as profound as that seen in the IL-1R KO mice (FIG. 9B).

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

We claim:

1. A method for treating or ameliorating hypertension comprising administering an IL-1 receptor antagonist at a therapeutically effective level to a subject with elevated blood pressure.

2. The method of claim 1, wherein treating or ameliorating hypertension comprises decreasing blood pressure to a therapeutically effective level.

3. The method of claim 1, wherein the IL-1 receptor antagonist is Anakinra.

4. The method of any one of claim 1, wherein the subject is human.

5. A method for modulating IL-1 receptor activation in a subject with elevated blood pressure comprising administering an IL-1 receptor antagonist.

6. The method of claim 5, wherein modulating IL-1 receptor activation comprises decreasing blood pressure to a therapeutically effective level.

7. The method of claim 5, wherein the IL-1 receptor antagonist is Anakinra.

8. The method of claim 5, wherein the subject is human.

9. A method of modifying IL-1 receptor signaling to treat hypertension, the method comprising administering an IL-1 receptor antagonist.

10. The method of claim 9, wherein modifying IL-1 receptor signaling comprises decreasing blood pressure to a therapeutically effective level.

11. The method of claim 9, wherein the IL-1 receptor antagonist is Anakinra.

12. A composition comprising an IL-1 receptor antagonist that decreases blood pressure to a therapeutically effective level in a subject with hypertension.

13. The composition of claim 12, wherein the IL-1 receptor antagonist is Anakinra.
